(19) United States
(12) Patent Application Publication
(10) Pub. No.: US 2023/0320623 A1
(43) Pub. Date: Oct. 12, 2023

(54) BREATH COLLECTOR AND METHOD FOR DIAGNOSIS AND/OR MONITORING

(71) Applicants: University of Tennessee Research Foundation, Knoxville, TN (US); UT-Battelle, LLC, Oak Ridge, TN (US)

(72) Inventors: Scott Strome, Memphis, TN (US); Elizabeth Fitzpatrick, Germantown, TN (US); Scott Retterer, Oak Ridge, TN (US); Rigoberto Advincula, Oak Ridge, TN (US)

(21) Appl. No.: 18/023,529

(22) PCT Filed: Aug. 25, 2021
(86) PCT No.: PCT/US2021/047547

§ 371 Date: Feb. 27, 2023

Related U.S. Application Data
(60) Provisional application No. 63/070,076, filed on Aug. 25, 2020.

(51) Int. Cl. A61B 5/097

(52) U.S. Cl. CPC A61B 5/097

(57) ABSTRACT

A device for capturing exhaled breath of an individual for diagnosis, monitoring, and/or study of conditions and/or diseases comprises a mouthpiece; a mixing chamber, to which the mouthpiece is connected; an exhaust tube connected to the mixing chamber, the exhaust tube having an outlet formed therein; and a container having a capture material disposed therein. The mixing chamber is formed such that the exhaled breath from the individual, which is introduced therein via the mouthpiece, contacts the capture material and traps an exhalant of interest in and/or on the capture material.

### CROSS-REFERENCE TO RELATED APPLICATIONS

This application claims benefit of and priority to U.S. Provisional Patent Application Ser. No. 63/070,076, filed Aug. 25, 2020, the contents of which are incorporated by reference herein in their entirety.

### STATEMENT OF GOVERNMENT SUPPORT

The United States Government has rights in this invention pursuant to contract no. DE-AC05-00OR22725 between the United States Department of Energy and UT-Battelle, LLC.

### TECHNICAL FIELD

The presently disclosed subject matter relates to a device suitable for the non-invasive respiratory collection of specimens (e.g., containing microbial and/or viral particles) from a subject. More particularly, the presently disclosed subject matter relates to devices, systems, and methods of collecting specimens for disease diagnosis and/or monitoring.

### BACKGROUND

The global pandemic caused by the novel coronavirus (SARS-COV-2) continues to take a tremendous toll on individual's physical and mental wellbeing, as well as causing devastating economic impacts on both national and global scales. At present, the diagnostic testing capabilities require multiple hours, and typically days, in order to perform the diagnostic procedure and report the result (e.g., whether a subject is infected). Given the fact that it is known that many infected individuals may be infectious (e.g., able to transmit viral particles to others) while asymptomatic and/or pre-symptomatic, the need to perform widespread diagnostic testing to detect infections in individuals before they are able to spread the virus to a significant number of others is paramount. At present, the long lead-times and scarcity of raw materials is a significant impediment to widespread diagnostic testing and the prevalence of SARS-COV-2 in many countries requires a large volume of testing to be performed in order to be able to effectively quarantine individuals who are infected and lower the prevalence of the virus in the community at large. As such, there presently exists a need to rapidly and accurately diagnose infected individuals.

Rapid economical diagnostic testing that could be employed almost universally by potentially infected individuals would allow for early quarantine of infected individuals, thereby preventing viral spread while also allowing for earlier medical interventions (e.g., monitoring, sensing, and/or treatment) for such infected individuals. One goal of the presently disclosed subject matter is thus to provide an approach for potentially infected individuals to collect specimens of exhaled breath for use in diagnostic assays, rather than conventionally used devices and methods. The presently disclosed subject matter is particularly advantageous in that no, or at least minimal, medical expertise is needed for collecting a specimen (e.g., in an “at home” environment) especially in children and/or the elderly.

In addition, there is a long-standing need for collection of exhalants for the diagnosis and/or monitoring of acute or chronic diseases. Diagnosis of some of these diseases such as Streptococcal infection and mononucleosis require uncomfortable pharyngeal swabs, in which the accuracy of the test result is operator dependent. It is envisioned that this device can replace these swabs, improving the patient's experience and the reproducibility of results. In other acute conditions such as alcohol intoxication, this device may prove more effective than existing technologies, as it will provide a tangible record of alcohol levels which can potentially be validated by an external party, if challenged. In addition, there is an emerging need for evaluation of metabolic disorders and the microbiome in the upper respiratory tract for which this device could facilitate specimen acquisition. Finally, there are other diseases and/or conditions in which capture of particulate exhalants present in exhaled breath are thought to be useful for diagnosis including, but not limited to, malignancies, microbiome, and DNA/RNA/protein capture and/or analysis.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the methods and devices disclosed herein are capable of collecting specimens for transport and transfer to another method of testing and diagnosis in another facility or laboratory. Optionally, the device may be capable of signifying the presence of pathogens in the specimen through a visual read out or sensor within the device itself, or through immediate application of the specimen to a secondary point-of-care testing device which offers such a read out.

According to an example embodiment, a device for capturing exhaled breath of an individual, the device comprising: a mouthpiece; a mixing chamber, to which the mouthpiece is connected; an exhaust tube connected to the mixing chamber, the exhaust tube comprising an outlet; and a container having a capture material disposed therein; wherein the mixing chamber is configured to cause exhaled breath from the individual introduced therein via the mouthpiece to contact the capture material and trap an exhalant in and/or on the capture material.

In some embodiments of the device, the exhalant comprises an infectious agent and/or a particle thereof.

In some embodiments of the device, the infectious agent is a virus, a bacterium and/or a fungus.

In some embodiments of the device, the exhalant comprises a protein, polynucleotide, lipid, carbohydrate, and/or chemical biomarker indicating a presence of a disease, optionally the disease comprising a cancer or a metabolic disorder.

In some embodiments of the device, the exhalant comprises an analyte indicating a presence of alcohol, tobacco, and/or other drugs.

In some embodiments of the device, the container is mechanically and/or adhesively connected to the mixing chamber, optionally in a removable manner.

In some embodiments of the device, the container is connected to the mixing chamber via a hinge.

In some embodiments of the device, the capture material is directly linked to a detection assay.

In some embodiments of the device, the detection assay comprises a colorimetric assay or a lateral flow assay.

In some embodiments, the device comprises: a flow restrictor positioned within the exhaust tube at a position adjacent to the outlet; and a resonator connected to the exhaust tube at the outlet, the resonator comprising a resonator chamber that is shaped such that, as a flow of the exhaled breath through the exhaust tube is restricted by the flow restrictor and is emitted from the outlet, the device is configured to produce an audible tone when a predetermined volumetric flow rate of the exhaled breath is emitted from the outlet.

In some embodiments of the device, the resonator chamber is closed by an end cap at an end of the resonator opposite the outlet.

In some embodiments of the device, the mouthpiece is removably connected to the mixing chamber; the exhaust tube is removably connected to the mixing chamber; and/or the resonator is removably connected to the exhaust tube.

In some embodiments of the device, the mouthpiece, the mixing chamber, and the exhaust tube are formed integrally with each other, as a monolithic structure.

In some embodiments of the device, the resonator is formed integrally with the exhaust tube, as a monolithic structure.

In some embodiments of the device, the capture material comprises a gel or a hydrogel.

In some embodiments of the device, the capture material is configured to release the exhalant captured therein for further diagnostic testing and/or the capture material is configured for release from the container.

In some embodiments of the device, the capture material comprises a gel of one or more of agarose, carbomers, Polyvinylalcohol (PVA), and polyacrylic acid (PAA).

In some embodiments of the device, the capture material is a viscous fluid.

In some embodiments of the device, the capture material is fixed onto an interior surface of the container in a manner of a coating on the interior surface of the container.

In some embodiments of the device, the device is made via an additive manufacturing technique.

In some embodiments of the device, the mouthpiece, mixing chamber, and exhaust tube are manufactured as a unitary structure via an injection molding technique.

In some embodiments of the device, the capture material comprises an electric charge to increase capture of the exhalant by the capture material, relative to a non-electrically charged capture material; the capture material is hydrophobic or hydrophilic to modify capture properties of the capture material; the capture material is molded to increase a surface area of the capture material that is exposed to the exhalant within the device; and/or the capture material is in a form of a ball inside the device.

In some embodiments of the device, the mixing chamber comprises an inner surface that is hydrophobic and/or negatively charged to inhibit the exhalant from adhering to the inner surface of the mixing chamber and to increase an amount of the exhalant that is captured by the capture material.

In some embodiments of the device, the exhalant comprises SARS-COV-2 virus and/or particles thereof, an infectious agent or particles thereof associated with a Streptococcal infection, and/or an infectious agent or particles thereof associated with mononucleosis.

In some embodiments of the device, the device is configured for diagnosis, monitoring, and/or study of conditions and/or diseases.

According to another example embodiment, a system for detecting an exhalant contained within exhaled breath of an individual, the system comprising at least one device as described herein

In some embodiments, the system comprises a device, container, and reagent for recovering the capture material and/or for recovering the exhalant from the capture material and, optionally, a plurality of single-use containers, mouthpieces, and/or exhaust tubes.

According to another example embodiment, a diagnostic test kit for detecting an exhalant contained within exhaled breath of an individual, the diagnostic test kit comprising at least one device as described herein.

In some embodiments, the diagnostic test kit comprises a device, container, and reagent for recovering the capture material and/or for recovering the exhalant from the capture material and, optionally, a plurality of single-use containers, mouthpieces, and/or exhaust tubes.

According to another example embodiment, a method for detecting an exhalant contained within exhaled breath of an individual, the method comprising: providing at least one device as described herein; flowing exhaled breath from the individual through the device for a predetermined period of time; and capturing the exhalant in the capture material.

In some embodiments, the method comprises producing an audible tone from the device when a sufficient volumetric flow rate of the exhaled breath is emitted through the outlet of the device.

In some embodiments, the method comprises measuring a duration of the audible tone to determine that the sufficient volumetric flow rate of the exhaled breath is provided to the device for at least the predetermined period of time.

In some embodiments, the method comprises removing the container from the mixing chamber and performing one or more further diagnostic testing techniques to detect whether the exhalant is contained within the capture material after the individual has provided the sufficient volumetric flow rate of the exhaled breath into the device for the predetermined period of time.

In some embodiments of the method, the one or more further diagnostic testing techniques comprise a colorimetric assay or a lateral flow assay.

In some embodiments of the method, the one or more further diagnostic testing techniques comprise polymerase chain reaction (PCR).

Accordingly, it is an object of the presently disclosed subject matter to provide devices, systems, and methods for detecting an exhalant contained within the breath of an individual. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

FIG. 1 is an external view of a device according to a first example embodiment for collecting a specimen according to the disclosure herein.

FIG. 2 is an isometric external view of a portion of a device according to a second example embodiment for collecting a specimen according to the disclosure herein.

FIG. 3 is a cross-sectional rear view of the device of FIG. 2 according to the disclosure herein.

FIG. 4 is a second cross-sectional side view of the device of FIG. 2, the plane in which the cross-sectional view is shown in FIG. 4 being orthogonal to the plane in which the cross-sectional view is shown in FIG. 3, according to the disclosure herein

FIG. 5 is an isometric external view of a portion of a device according to the second example embodiment, the portion shown in FIG. 5 being a container suitable for connection to the portion of the device shown in FIG. 2 to collect a specimen in the container, according to the disclosure herein.

FIG. 6 is a side view of the container shown in FIG. 5, with an example capture material disposed therein, according to the disclosure herein.

FIG. 7 is an external side view of the device according to FIG. 2, taken from the direction opposite the view shown in FIG. 4, schematically showing an example of the air flow pattern of the device during specimen collection, according to the disclosure herein.

FIG. 8 is an external front view of the device according to FIG. 2, according to the disclosure herein.

### DETAILED DESCRIPTION

The presently disclosed subject matter will now be described more fully with reference to the accompanying figures. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of temperature, time, weight, volume, concentration, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of, in one example ±20% or ±10%, in another example ±5%, in another example ±1%, and in still another example ±0.1% from the specified amount, as such variations are known to be appropriate to perform the disclosed methods. Additionally, the term “substantially” includes not only the specified amount, but can include, for example, ±20%, ±15%, ±10%, ±5%, ±1%, and ±0.5%, as may be readily understood by those having ordinary skill in the art.

Numerical ranges recited herein by endpoints include all numbers and fractions subsumed within that range (e.g. 1 to 5 includes, but is not limited to, 1, 1.5, 2, 2.75, 3, 3.90, 4, and 5).

The example devices and methods disclosed herein are useful in capturing particulate exhalants present in exhaled breath that are useful for diagnosis including, but not limited to, malignancies, microbiome, and DNA/RNA/protein capture and/or analysis.

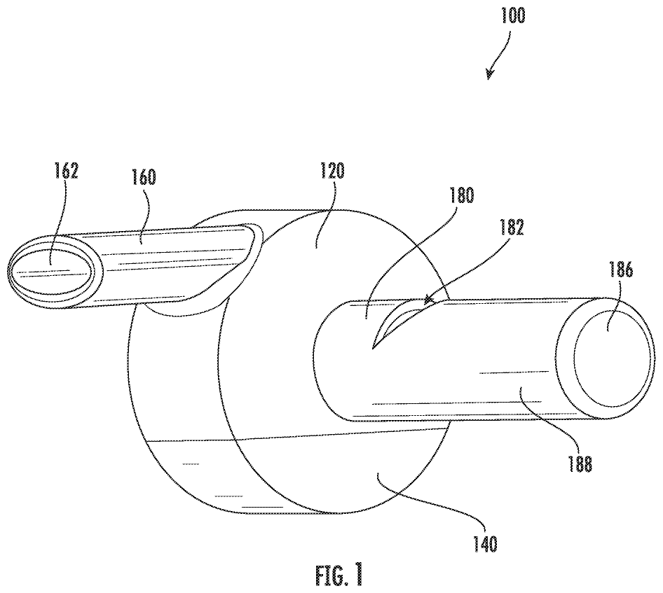

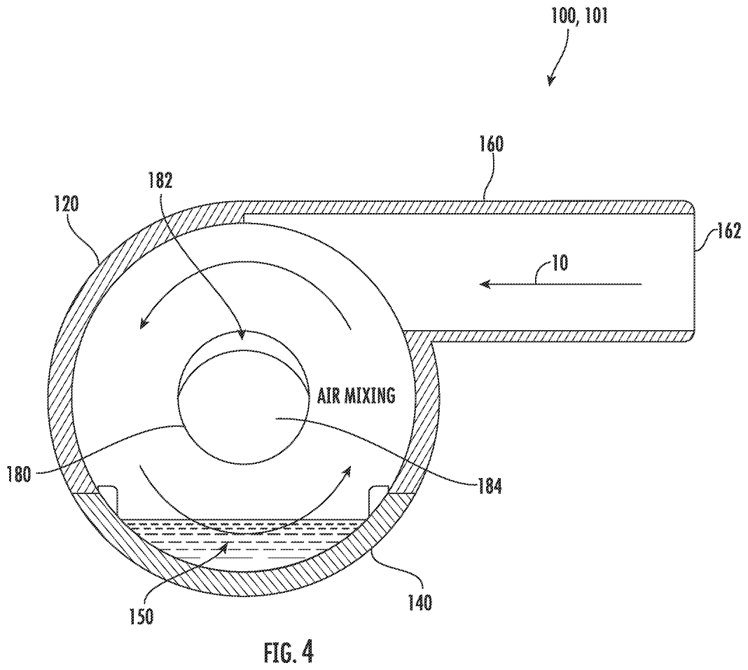

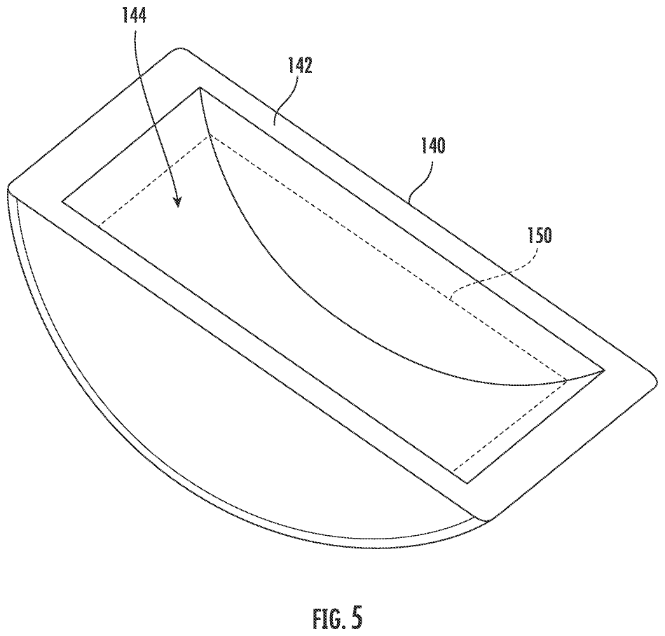

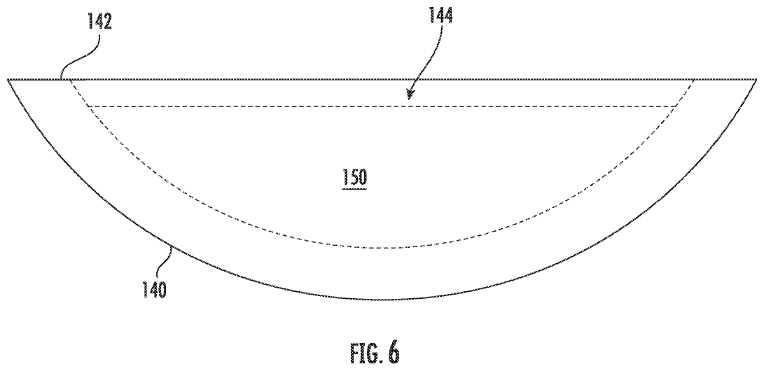

FIGS. 1 and 4-7 show various aspects of an example embodiment of a device, generally designated 100, for collecting any exhalants or analytes (e.g., infectious agent particles, such as but not limited viral particles, bacteriophage particles, prion particles, microbial particles, such as bacterial and/or fungal microbes, or other pathogens, alcohol, tobacco, and/or other drugs, and/or proteins, polynucleotides, lipids, carbohydrates, and/or nucleic acids associated with diseases, including cancer, metabolic disorders, etc., as well as the microbiome of the individual from which the specimen was obtained) contained in the exhaled breath of an individual (e.g., a human being or any suitable living creature capable of utilizing the device 100). As used herein, the term “biomarker” refers to any exhalant, which is contained within the breath of a person having a disease, in which the presence of the exhalant is an indicator that the person has the disease. The term “infectious agent” includes any infectious agent as would be apparent to one of ordinary skill in the art upon a review of the instant disclosure, such as but not limited to viruses, prions, and/or microbes. The terms “microbe” and “microbial” can, as used herein, be used to refer to any microbe as would be apparent to one of ordinary skill in the art upon a review of the instant disclosure, including, but not limited to, bacteria, fungi, other protists, and/or archaea. As used herein, the term “analyte” can be used to refer to a molecule, macromolecule, and/or chemical of interest in the form of released and/or detectable concentration of a bound ingredient within the specimen.

The device 100 comprises a mouthpiece 160, into which the potentially infected individual blows his/her exhaled breath for a prescribed period of time through an inlet 162 of the mouthpiece 160, a mixing chamber 120, to which a container 140 having a capture material 150 disposed therein is attached (e.g., preferably removably attached), and an exhaust tube 180 connected to the mixing chamber 120. The sidewall thickness of each component of the device 100 is advantageously designed to be robust, preventing delamination, cracking, or splitting thereof during normal use, or even upon excessive biting of, for example, the mouthpiece 160 by a person while blowing into the device 100 for the prescribed duration of time, which can be in a single exhalation and/or cumulative.

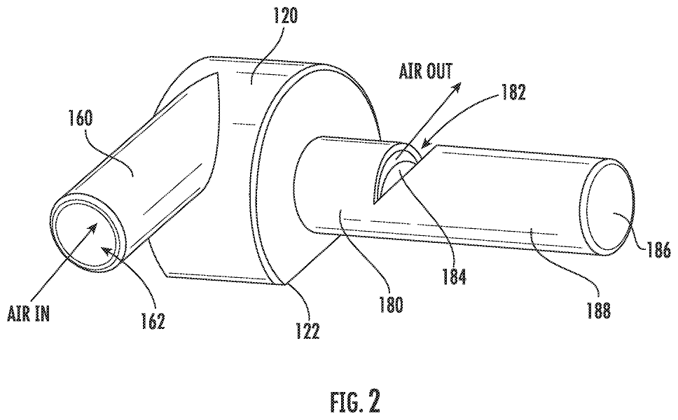

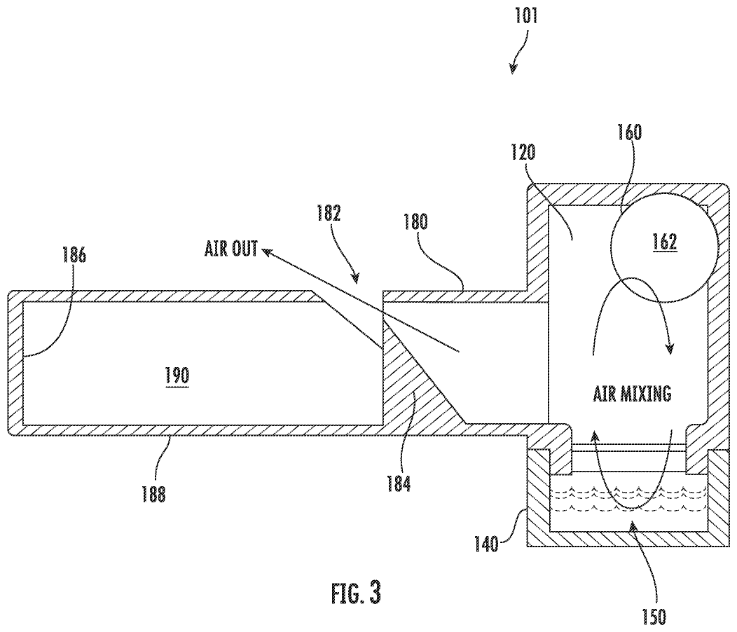

The mouthpiece 160 may have any suitable shape and/or length and may be made from any manufacturable composition of matter (e.g., polymers, plastic, thermoset, ceramic, metal, and the like). For example, in the embodiment shown in FIG. 1, the mouthpiece 160 and inlet 162 have a generally elliptical cross-sectional shape, which is substantially constant along the length thereof (e.g., in the axial direction of the mouthpiece 160, between the inlet 162 and the point where the mouthpiece 160 is joined to the mixing chamber 120). Another example embodiment of a device, generally designated 101, for collecting any exhalants or analytes contained in the exhaled breath of an individual is shown in FIG. 3, in which the general construction of the device 101 is substantially similar to that of the device 100, but in which the mouthpiece 160 and the inlet 162 have a cross-sectional shape that is generally circular, which is substantially constant along the length thereof, such that the mouthpiece 160 of the device 101 is generally in the shape of a hollow cylindrical prism, but having an end opposite the inlet 162 that terminates at the sidewall of the mixing chamber 120. A portion of the device 101 is shown in FIG. 2, including all portions that are integrally (e.g., monolithically, in a unitary manner) formed with the mixing chamber 120, with the container 140 of the device being omitted from the view shown in FIG. 2. In some embodiments, the cross-sectional shape and/or dimensions of the mouthpiece 160 may vary along the length thereof, between the inlet 162 and the intersection between the mouthpiece 160 and the outer circumferential wall of the mixing chamber 120. As used herein, “intersection” can mean, for example, the point, plurality of points, surface, and the like where the mouthpiece 160 is connected to the mixing chamber 120.

By way of example and not limitation, the cross-sectional shape of the mouthpiece 160 can change from being generally elliptical at the inlet 160 to generally circular where the mouthpiece 160 intersects with the mixing chamber 120. Similarly, the cross-sectional shape (e.g., circular, elliptical, or any suitable shape) of the mouthpiece 160 can remain the same (e.g., constant, allowing for variations due to manufacturing tolerances) along the length thereof, but can, for example, be tapered so that the inlet 162 has a cross-section that is larger or smaller than the cross-section of the mouthpiece 160 at the location where the mouthpiece 160 connects to and/or intersects with the mixing chamber 120. In some embodiments, the cross-sectional shape and/or dimensions of the mouthpiece 160 can vary (e.g., continuously and/or in a step-wise manner) along the length thereof. The various aspects of the mouthpiece 160 disclosed herein can be combined to form a mouthpiece 160 having substantially any shape.

It is advantageous for the mixing chamber 120 and the container 140 to form, other than the locations where the mouthpiece 160 and the exhaust tube 180 are attached thereto, a substantially enclosed region in which the breath exhaled by the individual is impinged upon, or otherwise comes into contact with (e.g., due to turbulence of the air flow path 10 within the mixing chamber 120), the capture material 150 contained within the container 140. The bottom edge of the mixing chamber 120 comprises a mating surface 122 for assembly with the mating surface 142 defined by the top, or upper, surface of the container 140. Thus, the region in which the exhaled breath is introduced against the capture material 150 is defined by assembling the mixing chamber 120 to the container 140 at their respective mating surfaces 122, 142. The mating surfaces 122, 142 have a substantially similar profile to each other to allow an assembly of the mixing chamber 120 and the container 140. In some embodiments, a locking feature, such as a sliding, keyed, structure, may be provided in/on the mating surfaces 122, 142 to prevent the container 140 from accidentally being dislodged from the mixing chamber 120, such as during use.

The container 140 includes a cavity, generally designated 144, in which the capture material 150 is provided. Any suitable capture material 150 capable of collecting an exhalant (e.g., a sufficient quantity of an exhalant for a planned analysis; by way of further example, a desired or specified number of infectious agent particles, or other suitable analytes) that comes into contact the capture material 150 may be used. The capture material 150 can comprise a fluid and/or a solid. An example of a suitable capture material 150 can comprise agarose gel, which can be added to the device 100, 101 (e.g., in the container 140) prior to assembly of the container 140 with the mixing chamber 120 and subsequent use of (e.g., by exhaling into) the device 100, 101 by an individual. In embodiments where the capture material 150 comprises, consists essentially of, or consists of agarose gel, the agarose gel can be reconstituted, for example, with water and/or in an aqueous buffer (e.g., TBS, TAE, PBS, etc.) to form an aqueous solution (e.g., having 4% agarose concentration or any other desired concentration) or can be added directly within the container 140 in gel form. Other electrophoresis types of gels, natural gels, and/or commercially available synthetic gels may be used as well. The capture material can, in some embodiments, comprise a carbomer, which is a synthetic modified cellulose gel that is commercially available, with derivated crosslink acrylates. Carbomers may be reconstituted using water from a powder form.

In some embodiments, the capture material 150 can comprise, consist essentially of, or consist of synthetic polymers based on hydrogels that can be tuned through salts and solvent selectivity, examples of which can include polyvinyl alcohol (PVA), polyacrylic acid (PAA), poly-N-isopropyl acrylamide (P-NIPAM), and other vinyl polymer based hydrogels that are capable of physical crosslinking and control of viscoelastic behavior based on molecular weight and solvent quality. The molecular weight can range from, in some embodiments, thousands of daltons to hundreds of thousands of daltons or grams/mol, the solubility can range from hundreds to thousands of mg/ml, and/or, as a percent weight basis, from about 1 to tens of a percentage by weight. An example, non-limiting composition of such a capture material 150 can have polymers that are either chemically (e.g., covalently, with elemental C—C, C—O, C—N, C—S bonding) or physically (e.g., ionic, H-bonding, chelating, etc.) cross-linked. In some embodiments, the capture material 150 comprises, consists essentially of, or consists of non-vinyl polymers, such as polylysine, polyethyelene imine (PEI), etc., based on anionic and cationic or polyelectrolyte pH and/or salinity control thereof.

In some embodiments, the capture material 150 comprises, consists essentially of, or consists of chemically accessed in-situ polymerized hydrogels, which can comprise, consist essentially of, or consist of polyethylene glycol acrylated (PEGDA) mixed with various ratios of di-acrylate crosslinkers (e.g., ethylene glycol dimethacrylate (EGDMA) and a benzophenone type photoinitiator). Such types of a capture material 150 require photopolymerization with UV light and may be advantageous for use in such a device 100, 101 due to their ability to be chemically fixed (e.g., coated) on the inner surface of the device 100, 101, which can sometimes be referred to as a “whistle,” such as on the inner surface of the container 140, thereby advantageously avoiding any restrictions on orientation of the device 100, 101 during manufacture, transport, use, analysis, and the like.

The capture material 150 can comprise, consist essentially of, or consist of gels, hydrogels, and/or crosslinked gel modifiers to capture an exhalant of interest (e.g., infectious agents and particles thereof, including viral particles, such as for the SARS-COV-2 virus) within the device 100, 101. In such instances, the capture material 150 acts as the capture matrix and traps the exhalant (e.g., virus or other analyte of interest) through non-covalent interactions (e.g., hydrogen (H)-bonding, dipole forces, ionic interactions, etc.) of the exhalant with the capture material 150. In some embodiments, the particular composition of the capture material 150 is based on exhalant molecules and macromolecules having a functionality of 3 or more and is capable of physical crosslinking and/or chemical crosslinking. Gels suitable for use as the capture material 150 can be based on natural sources (e.g., polysaccharides, glycoproteins, etc.) or synthetic sources (e.g., acrylate, amine, etc.) having the ability to form a network, an interpenetrated network (IPN), and/or a semi-interpenetrated network (SIPN). The degree of crosslinking and type of crosslinking (e.g., chemical and/or physical) of such gels determines the viscoelastic nature of the capture material 150 and, necessarily, the ability of the capture material 150 to maintain its form and to be stable in the presence of water (e.g., as in a hydrogel).

In some embodiments in which the capture material 150 comprises, consists essentially of, or consists of gel(s), it can be advantageous for the gel(s) to comprises, consists essentially of, or consists of a hydrogel, which is swelled and/or reconstituted with hydration (e.g., by adding water) in forming the capture material 150 and/or as or when the capture material 150 is being provided within the container 140. In some embodiments, the capture material 150 can comprise, consist essentially of, or consist of non-hydrogel matrices or hybrid-hydrogel matrices that function to capture the exhalant of interest (e.g., an infectious agent or particles thereof), while still allowing the exhalant of interest to be capable of detection and/or to be released from the capture material 150, such as, for example, during subsequent diagnostic testing, such as in a lateral flow assay. In some embodiments, the capture material 150 is configured for release from the container 140.

In some embodiments in which the capture material 150 comprises gel(s), the gel(s) can be suitable for blending and/or co-polymerization to: (1) capture and trap the exhalant of interest (e.g., an infectious agent and/or particles thereof) from the air flow path 10 through the device; (2) capture and release the exhalant of interest (e.g., an infectious agent and/or particles thereof); (3) preserve or degrade the exhalant of interest (e.g., an infectious agent and/or particles thereof) as a capture species; (4) provide in situ-diagnosis or detection of the exhalant of interest (e.g., an infectious agent and/or particles thereof) in the presence of bio-markers or sensor elements that can transduce by absorption, fluorescence, change in electrical or dielectric properties, etc.; (5) allow for use as a capture matrix for sensor-device fabrication (e.g., as a sensor recognition element); and (6) contain the exhalant of interest (e.g., an infectious agent and/or particles thereof) on the surface of the capture material 150 for safe-handling resources. In some embodiments, such gels are based on commercially available gels (e.g., agarose, carbomers, Polyvinylalcohol (PVA), polyacrylic acid (PAA), etc.), where the gel is loaded on and/or in the container 140 of the device 100, 101 in dry and/or hydrated form(s) to provide optimum capture of the exhalant of interest (e.g., an infectious agent and/or particles thereof) from impingement and/or contact of the air flow path 10 onto the capture material 150.

In some embodiments in which the capture material 150 comprises gel(s), the gel(s) can be made via in-situ photo-polymerization or crosslinking with the use of specific monomers, crosslinkers, and photoinitiators (e.g., polyethylene glycol acrylated (PEGDA) of various molecular weights (MW), mixed with various ratios of di-acrylate crosslinkers, e.g. ethylene glycol dimethacrylate (EGDMA) and a benzophenone type photoinitiator).

In some embodiments wherein the capture material 150 comprises gel(s), the gel(s) can host other components which are classified as: (1) rheology modifiers (e.g., viscoelatic modifiers)—other polymers or nanoparticles; (2) stabilizers (e.g., absorption, salt chelation, preservatives, surface silanes, etc.); (3) degradation agents (e.g., oxidizing agents, catalysts); (4) biomarkers (e.g., proteins, polynucleotides, lipids, carbohydrates, enzymes, bio-conjugated polymers, chemicals, etc.) that can signal specific adsorption or binding events; (5) absorption or fluorescent dyes and nanoparticles that produce an optical event which can be enhanced or attenuated, but are otherwise detectable by optical and/or spectroscopic methods; and (6) other additives that enable physico-chemical function with or without the presence of the exhalant of interest.

In some embodiments in which the capture material 150 comprises gel(s), the gel(s) may be “fixed” utilizing “grafting onto” and “grafting from” approaches that result in the gel(s) being fixed on the surface of the device 100, 101 (e.g., on the inner surface of the container 140) and produces robustness and/or stabilization with covalent or strong-non-covalent interaction, preserving the gel adsorption on the surface of the device 100, 101. This can be done by the gel(s) being “grafted onto” the surface of the device 100, 101 with both morphological modification of the device 100, 101 (e.g., micro- to nano-roughness or patterning level) and/or with chemical reactive groups. In such embodiments, the gel(s) is/are bound to the surface of the device 100, 101 by adhesive forces that cause the gel to stay (e.g., be “fixed”) in one location (e.g., so as to not be displaceable within the device 100, 101 depending on the orientation of the device 100, 101 relative the direction of the gravity vector). In some other example embodiments, the gel(s) can be “grafted from” the surface of the device 100, 101 through in-situ polymerization or crosslinking. According to such embodiments, the surface of the device 100, 101 will have a fixed density of the photo-initiators or reactive silanes, isocyanates, or aldehydes; the gel(s) will then form by initiating the photopolymerization or the crosslinking on the surface of the device 100, 101. Such “grafting” techniques used in conjunction with a capture material 150 comprising a gel can advantageously enable controlled adhesion on the surface of the device 100, 101 and/or surface modification of the device 100, 101 based on in-situ deposition chemistry. The monomer sequence, the MW, the MW distribution, and/or the degree of crosslinking of the capture material 150 are controllable based on the surface chemistry of the device 100, 101. Thus, the surface chemistry of the internal surface of the container 140 may be different from the surface chemistry of the internal surface of the mixing chamber 120, for example. In some embodiments, the inner surface of the mixing chamber, either entirely or partially, is treated and/or coated with a hydrophobic material and/or is negatively charged so that less of the contents of the exhaled breath would adhere onto the internal surface of the mixing chamber 120 to increase the amount of the contents of the exhaled breath that accumulated on and/or in the capture material 150 of the container 140.

According to embodiments in which the capture material 150 comprises gel(s), the gel(s) can comprise natural or synthetic polymers or hybrids which can be controlled in terms of MW, degree of cross-linking, nature of hydration, and can be prepared ex-situ or in-situ. The components of such capture material 150 can therefore be classified as pure gel, IPN, and/or SIPN in the presence of other polymers. The gel(s) can be used directly in hydrated form or blended with other additives. The gel(s) can be polymerized or crosslinked in situ via photopolymerization. The gel(s) can be surface bound on the device 100, 101 (e.g., on the inner surface of the container 140) by either a “grafting onto” or a “grafting from” technique. Such gel-based capture material 150 types are operable to capture the exhalant of interest (e.g., an infectious agent and/or particles thereof) and preserve the function of the surface of the device 100, 101 on which the capture material 150 is provided for collection and release of such an exhalant of interest (e.g., an infectious agent and/or particles thereof) for further diagnostic testing, when necessary and/or desired. The gel(s) are thus configured as a temporary collection matrix for the exhalant of interest, which will be isolated or recovered from the capture material 150 for further testing (ELISA, PCR, GC/MS, etc.). Such capture material 150 comprising gel(s) can capture the exhalant of interest (e.g., an infectious agent and/or particles thereof), such that the exhalant of interest is bound on the surface of the capture material 150 for direct testing (ELISA, PCR, GC/MS, etc.). The exhalant of interest (e.g., an infectious agent and/or particles thereof) captured by the capture material 150 can therefore be detected directly on the surface of the device 100, 101 (e.g., on the inner surface of the container 140, in the manner of a substrate formed thereon by the capture material 150) through spectroscopic or other sensor transduction methods. In such embodiments, the exhalant of interest (e.g., an infectious agent and/or particles thereof) captured by the capture material 150, as well as the gel(s) of the capture material 150, are configured as a sensor element.

In some embodiments, the capture material 150 comprises an electric charge to increase capture of the exhalant by the capture material 150, relative to a non-electrically charged capture material 150. In some embodiments, the capture material 150 is hydrophobic or hydrophilic to modify capture properties of the capture material 150. In some embodiments, the capture material 150 is molded to increase a surface area of the capture material 150 that is exposed to the exhalant within the device 100, 101. In some embodiments, the capture material 150 is in a form of a ball inside the device 100, 101. These features can be combined without limitation. For example, the capture material 150 comprises an electric charge to increase capture of the exhalant by the capture material 150, relative to a non-electrically charged capture material 150; the capture material 150 is hydrophobic or hydrophilic to modify capture properties of the capture material 150; the capture material 150 is molded to increase a surface area of the capture material 150 that is exposed to the exhalant within the device 100, 101; and/or the capture material 150 is in a form of a ball inside the device 100, 101.

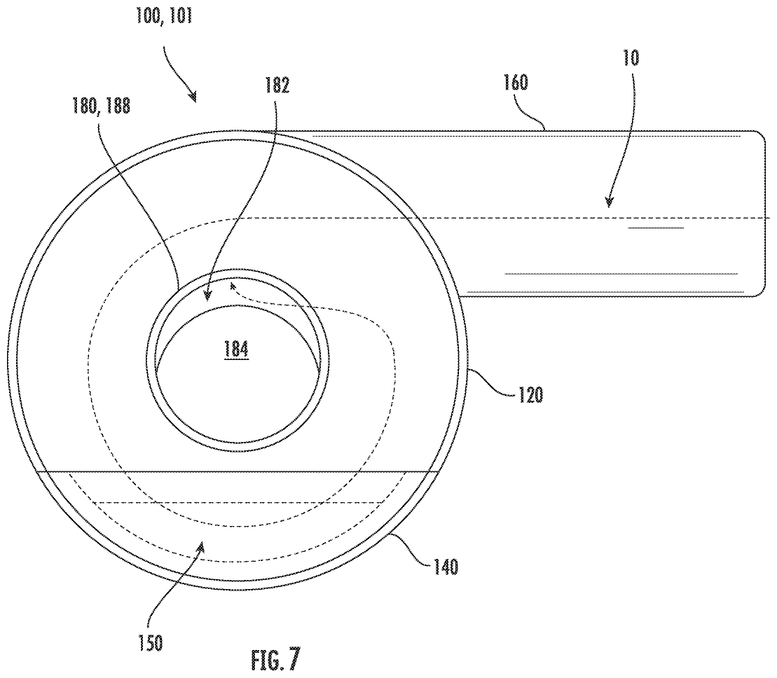

It is advantageous for the mixing chamber 120 to have a substantially cylindrical shape, in which the diameter can be greater than, smaller than, or the same size as the thickness, which is measured in a direction perpendicular to the plane in which the diameter of the mixing chamber 120 is defined, and for the mouthpiece 160 to be attached to the mixing chamber 120 at a position on or about the outer circumferential surface of the mixing chamber 120, such that the exhaled air introduced through the mouthpiece 160 by the individual enters the mixing chamber 120 in a direction that is substantially tangential to the inner surface of the mixing chamber 120, which can be seen in FIG. 7, for example. Thus, the air flow path 10 is defined as entering the mixing chamber 120 via the mouthpiece 162, flowing around the internal surface of the mixing chamber 120, impinging or otherwise contacting the capture material 150 within the container 140, and ultimately exiting the mixing chamber via the exhaust tube 180, which can be achieved due to positive pressure within the mixing chamber 120 due to continued exhalation into the mouthpiece 160 by the individual and/or due to a flow-directing structure, such as fins or a wall, that restrict or block entirely re-entrainment of recirculated air within the mixing chamber 120 into the air flow path 10 as the exhalant enters the mixing chamber 120 from the mouthpiece 160. In some embodiments, it may be advantageous to provide internal flow directing structures (e.g., a helical structure, which can fully span the entire diameter of the mixing chamber, or extend radially inwardly from the internal surface of the mixing chamber by a distance less than the radius of the mixing chamber 120, such that a central region of the mixing chamber 120 remains hollow) within the chamber to impinge the exhaled breath against the capture material a prescribed number of times. In some such embodiments, the internal flow directing structures may be formed such that the mouthpiece 160 is only connected to a single pathway defined by the internal flow directing structures, such that the air flow path follows in a sequential manner through the internal flow directing structures to ensure that exhalant is impinged upon the contact medium a prescribed number of times before exiting the mixing chamber 120 through the exhaust tube 180. In some embodiments, one or more of the internal surfaces of the mixing chamber can be provided with one or more flow-disrupting structures (e.g., structures that increase turbulent flow, such as is defined by Reynold's number), such as on the inner circumferential and/or vertical walls thereof, to increase the interaction of the contents of the exhaled breath with the capture material 150. A nonlimiting example embodiment of such turbulence-inducing features include one or more ridges, or protrusions, that extend inwardly (e.g., radially) from the inner surface of the mixing chamber 120 and are inclined at an angle, or perpendicular, relative to the air flow path 10. Another nonlimiting example embodiment, one or more pedestals (e.g., having any suitable cross-sectional shape and extending inwardly from the inner surface of the mixing chamber 120) may be provided to impinge on, or otherwise extend into, the air flow path 10 to disrupt laminar flow.

The exhaust tube 180 is connected (e.g., in an integral, unitary, and/or monolithic manner) to the mixing chamber 120 and extends in a direction that is inclined at an angle relative to the direction of extension of the mouthpiece 160, such that the direction of extension of the exhaust tube is not tangential to the outer or inner surfaces of the missing chamber 120. In the example embodiment shown, the mixing chamber 120 has at least two substantially flat and/or planar surfaces that define the ends of the hollow cylindrical prism formed by the circumferential wall of the mixing chamber 120. The exhaust tube 180 is arranged such that the direction of extension of the exhaust tube 180 is perpendicular to the direction of extension of the mouthpiece 160. In some embodiments, however, the surface to which the exhaust tube 180 is attached to the mixing chamber 120 can be planar, concave, and/or convex. In some embodiments, it is envisioned that the mixing chamber 120 can have a generally spherical shape (e.g., when assembled with the container 140) and the exhaust tube 180 can be attached to any suitable surface thereof that ensures adequate contact of the air flow path against the capture media 150 within the container 140, which can also have a shape of a portion of the sphere defined by the mixing chamber 120 and the container 140 when assembled together.

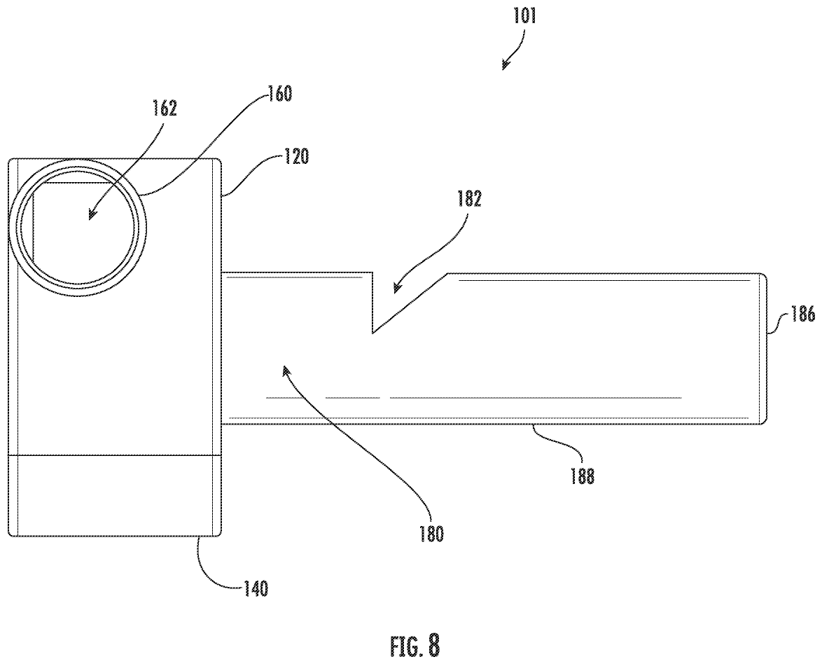

As shown, for example, in FIGS. 3 and 8, the exhaust tube 180 has an outlet, generally designated 182, formed on an outer (e.g., circumferential) surface thereof to complete the air flow path 10 for the exhaled breath introduced by the individual through the inlet 162 of the mouthpiece 160. It is advantageous for the devices 100, 101 to have a flow restrictor 184 positioned within the exhaust tube 180, adjacent to the outlet 182. The flow restrictor 184 is shown as being positioned within the exhaust tube 180 between the outlet 182 and the mixing chamber 182. The flow restrictor 184 is shown as having a generally angled shape, formed in cross-section by a generally vertically-extending wall and an angled wall, with the angled wall of the flow restrictor 184 forming an oblique angle with the inner surface of the circumferential wall of the exhaust tube 180, from which such flow restrictor 184 extends, as shown in FIG. 3. Thus, in cross-section taken along the longitudinal axis of the exhaust tube 180, the flow restrictor 184 has a generally triangular shape. In some embodiments, it may be advantageous, when forming the device 100, 101 using an additive manufacturing technique, to form the angled wall without the vertically-extending wall, in the manner of an inclined plate, such that the amount of material needed to form the flow restrictor 184 can be reduced. In the example embodiments shown, the angled wall of the flow restrictor 184 terminates coplanar with an edge of the outlet 182, such that no portion of the flow restrictor 184 extends beyond any portion of the outlet 182, in the longitudinal direction of the exhaust tube 180. As shown in FIG. 2, the end of the flow restrictor 184 adjacent to the outlet 182 has a generally curved shape, being attached to the circumferential walls of the exhaust tube 180 in the plane defined by the edge of the outlet 182 closest to the mixing chamber 120, but creating a crescent-shaped gap between the edge of the flow restrictor 184 and the inner surface of the exhaust tube 180 in the plane defined by the edge of the outlet 182 closest to the mixing chamber 120. According to such a structure of the flow restrictor 184, the effective diameter of the exhaust tube 180 is decreased at (e.g., coplanar with, or immediately before) the outlet 182 in the plane defined by the edge of the outlet 182 closest to the mixing chamber 120, along the longitudinal direction of the exhaust tube 180. In the example embodiment shown, the flow restrictor 184 has an outer profile that is generally in the shape of a right circular cone.

The exhaust tube 180 extends beyond the flow restrictor 184 and the outlet 182 is formed generally in the shape of a notch, extending into the exhaust tube 180. While the outlet 182 is a void, or hollow space, the shape of the void formed by the outlet is also generally in the shape of a right circular cone, or a portion thereof, in which the base of the void formed by the outlet 182 is substantially coplanar with (e.g., allowing for misalignments that may occur due to tolerances during manufacture of the device 100, 101) the base (i.e., the vertically-extending wall) of the flow restrictor 184. Thus, the void formed by the outlet 182 extends in the longitudinal direction of the exhaust tube 180, away from the flow restrictor 184, and the depth of the notch that defines the outlet 182 decreases (e.g., in a continuous manner, such as by having a constant slope) along the longitudinal direction of the exhaust tube 180. A resonator 188 is attached to the exhaust tube 180 (e.g., in an integral, unitary, or monolithic manner) at the outlet 182, such that the resonator 188 extends longitudinally beyond the outlet 182. The resonator 188 has a constant diameter and is in the shape of a hollow cylindrical prism and terminates at the end cap 186, such that the resonator 188 defines a substantially fully-enclosed resonance chamber 190, which extends coaxial with the exhaust tube 180 beyond the outlet 182, such that the device 100, 101 is configured to emit an audible tone when a predetermined volumetric flow of air (e.g., the exhalant from the individual, introduced within the device 100, 101 through the mouthpiece 160, which has already interacted with the capture material 150 within the container 140 and existed the mixing chamber 120) passes through the outlet 182.

The exhaust tube 180, the outlet 182, flow restrictor 184, and/or the resonator 188 may have any suitable shape and/or length based on the volumetric flow of exhaled breath that must be introduced into the mixing chamber 120 by the individual to ensure sufficient contact with the capture material 150 to enable collection of the exhalant of interest. Thus, the dimensions of the exhaust tube 180, the outlet 182, flow restrictor 184, and/or the resonator 188 may be selected such that the audible tone is only generated when a sufficient volume of air is passing through the outlet 182, the “sufficient volume of air” being the same as, or within a predefined range of, the volumetric flow of exhaled breath that must be introduced into the mixing chamber 120 by the individual to ensure sufficient contact with the capture material 150 to enable collection of the exhalant of interest.

In some embodiments, the exhaust tube 180 and the resonator 188 may have a cross-sectional shape and/or dimensions that are different from each other. In the example embodiments shown in FIGS. 1-8, the exhaust tube 180 and the resonator 188 are integrally formed (e.g., in a unitary, or monolithic manner) and have a generally circular cross-sectional shape, when viewed along the longitudinal axis of the exhaust tube 180, that has a same diameter along the length thereof. In some embodiments, the cross-sectional shape and/or dimensions of the exhaust tube 180 may vary along the length thereof, between the point at which the exhaust tube 180 is connected to the mixing chamber 120 and the outlet 182, and/or the cross-sectional shape and/or dimensions of the resonator 188 may vary along the length thereof, between the outlet 182 and the end cap 186.

By way of example and not limitation, the cross-sectional shape of the exhaust tube 180 and/or the resonator 188 can change along their respective lengths. Similarly, the cross-sectional shape (e.g., circular, elliptical, or any suitable shape) of the exhaust tube 180 and/or the resonator 188 can remain the same along the length thereof, but can, for example, be tapered. In some embodiments, the cross-sectional shape and/or dimensions of the exhaust tube 180 and/or the resonator 188 can vary (e.g., continuously and/or in a step-wise manner) along the respective lengths thereof. The various aspects of the exhaust tube 180 and/or resonator 188 disclosed herein can be combined to form an exhaust tube 180 and resonator 188 having substantially any cross-sectional shape, direction of extension, and/or length. In some embodiments, the resonator 188 is removably attached (e.g., by a threaded engagement) to the exhaust tube 180 at the outlet 182 to allow for resonators 188 of different cross-sectional shape, size, and/or length to be attached to the exhaust tube 180 at the outlet 182 to produce an audible tone of any desired frequency. In some embodiments, the resonator 188 can be configured to be polytonal when a predetermined volume of air is passing through the outlet 182, as this could advantageously aid in the proper volume of air being exhaled by an individual using the device 100, 101.

The design of the exhaust tube 180, outlet 182, flow restrictor 184, and/or resonator 188 to produce an audible tone when at least a prescribed volumetric flow rate is emitted through the outlet 182 is advantageous because it allows for, in the case of a self-administered or “at home” test, for a person (e.g., a person who is suspected of being infected with a contagious pathogen, for whom it may be undesirable to have physically present within a healthcare facility) to confirm that the individual is exhaling (e.g., blowing) a sufficient volumetric flow rate of air through the device 100, 101 to produce the audible tone. The audible tone is also advantageous for a person (e.g., a healthcare professional) collecting, or monitoring the collection of, a specimen from an individual using the devices 100, 101 disclosed herein because the audible tone provides feedback to confirm that the individual is exhaling a sufficient volume of air through the device 100, 101 to produce the audible tone. Correspondingly, the lack of an audible tone while the individual is exhaling into the device 100, 101 is an indicator to the individual and/or the healthcare professional that an insufficient volumetric flow rate of air is being exhaled into the device 100, 101 by the individual, which may result in inaccurate test results due to the collection of the specimen being faulty, such that corrective action can be undertaken to ensure proper specimen collection to allow for diagnosis of the individual.

The design of the device 100, 101 to produce an audible tone at a specified volumetric flow rate of air through the outlet 182 is also advantageous because it may be necessary, in order to properly collect a specimen, to have the individual blow into the device 100, 101 for a prescribed amount of time. As such, the duration of the audible tone can be used to determine that the individual provided a sufficient volume of exhaled breath into the device 100, 101 for a sufficient duration of time in order to assure that, if the individual were actually infected, or where otherwise positive for an analyte in the exhaled breath, a sufficient quantity of the infectious agent and/or analyte of interest would have been captured by the capture material 150 contained within the container 140 for further diagnostic testing, which can include, for example and without limitation, lateral flow assay, polymerase chain reaction, and the like.

In some embodiments, such as where the individual is suspected of being infected with an infectious pathogen, it can be advantageous for a filter to be provided within or external to the device 100, 101 to provide particulate filtration of the pathogen from the air emitted through the outlet 182, to protect people around the individual during use of the device 100, 101. Such a filter can be provided, for example, as a sleeve that can be inserted over and/or around the exhaust tube 180 and the resonator 188, such that the air that is emitted from the outlet 182 must pass through the filter. In some embodiments, a filter may be provided within, or at an entrance of, the exhaust tube 180, such that the pathogen can be removed from the air after sample collection simultaneously as the air exits the mixing chamber 120 into the exhaust tube 180.

The dimensions of all and/or some of the portions of the device 100, 101 may be selected based on the physiology and/or age of the potentially infected individual. For example, the dimensions of the mouthpiece 160, mixing chamber 120, container 140, exhaust tube 180, flow restrictor 184, outlet 182, and/or resonator 188 can be different to produce an audible tone for a volumetric flow rate of air that can be readily produced by a person with reduced lung capacity compared to an average adult (e.g., children and/or persons having reduced lung capacity). In some such embodiments, it may be necessary for such persons to produce the audible tone for a longer duration to ensure a same total volume of air passes through the device 100, 101 and comes into contact with the capture material 150 within the container 140. In order to account for the reduced lung capacity of such an individual, it may be permissible to measure an aggregate amount of time during which the audible tone is produced to allow for such individual to breathe between exhalations to produce a sufficient total volume of exhaled breath into the device 100, 101. In some embodiments, the exhaust tube 180 and/or the mouthpiece 160 can be integrally formed and/or removably attached to the mixing chamber 120 to allow for the components of the device 100, 101 to be sterilized and reused, as appropriate

Such devices 100, 101 as are disclosed herein can be made using any suitable manufacturing technique, including, for example, additive manufacturing (e.g., “3D printing”), formative manufacturing (e.g., injection molding, thermoforming, etc.), subtractive manufacturing (e.g., milling, polishing, etc.), and the like, based on whether the device 100, 101, other than the container 140, is a generally unitary (e.g., monolithically formed) structure or if portions of the device 100, 101 can be assembled together to form different configurations of the device 100, 101 based on the physiology of the individual who uses the device 100, 101.

An example embodiment of such a device 100 was produced using an additive manufacturing technique in a FormLabs 2 3D printer using the manufacturer's proprietary Rigid 1 resin as a representative device. The resin resembles a glass reinforced nylon and is mechanically and thermally stable. After printing, the devices 100 were rinsed in isopropyl alcohol (IPA) and UV cured at 80° C. to completely remove any residual solvents left behind during the printing and curing processes. A second IPA rinse and UV cure were performed both to ensure satisfactory structural rigidity and also to provide additional sterilization for the resultant devices 100. After being manufactured, the devices 100 were placed in a sealed autoclavable bag and autoclaved for sterilization. Prior to use, the devices 100 were removed from the autoclaved bag and the container of each device 100 was loaded with about 250 μl of fresh 4% agarose gel within a Class II Biosafety Cabinet and then either used or placed in a sealed bag, sterilized with ultraviolet (UV) light, and stored until ready for use.

In some embodiments, the components of the device 100, 101 are injection molded and the device 100, 101 is made in two, three, or more physically separate and discrete components, which are subsequently assembled mechanically and/or with adhesive to produce the devise 100, 101. The placement of seams and/or hinges of the device 100, 101 to allow for injection molding will be apparent to one of ordinary skill in the mechanical arts.

In some embodiments, the device 100, 101 as disclosed herein can be included within a system and/or diagnostic test kit. In embodiments in which the mouthpiece 160 and/or the exhaust tube 180 are removably attached to the mixing chamber 120, the system and/or test kit can include a plurality of mouthpieces 160 and/or exhaust tubes 180 having different geometries to allow for the system to be used for collecting a specimen from, for example, an adult or a child by assembling the components of a single system in a different configuration depending on whether it is intended for use by an adult or a child. In some embodiments, the system comprises a device, container, and reagent for recovering and/or analyzing the capture material and/or for recovering the exhalant from the capture material. In some embodiments, the diagnostic test kit comprises a device, container, and reagent for recovering and/or analyzing the capture material and/or for recovering the exhalant from the capture material.

In some embodiments, the system and/or test kit are configured for use with software stored and executed on a personal electronic device (PED), such as, for example and without limitation, a smart phone, a tablet, a personal computer, and the like. According to embodiments of the system and/or test kit, the application prompts a user to input configuration information regarding the device 100, 101 (e.g., a model number, which components of the kit are assembled together, if removable, and/or information regarding the individual that will use the device 100, 101, such as age). From this information, the application can determine the frequency of the audible tone and, using a microphone associated with (e.g., in electronic communication with, whether discrete from or integral to) the PED, record audio while the specimen is collected using the device 100, 101. Based on the configuration of the device 100, 101 and/or the physiology of the individual using the device 100, 101, the frequency of the audible tone and the duration of the predetermined period of time for which the audible tone must be produced (e.g., continuously or in aggregate) can be determined by the application. The audio detected and/or recorded by the microphone can be analyzed substantially contemporaneously (e.g., in real time, allowing for lags associated with data processing, typically on the order of milliseconds) and a timer can be initiated when an audible tone at the frequency preset by the software is detected in the audio signal. The timer can be stopped when the audible tone stops and either restarted from zero or from the last time value when the audible tone is detected again, based on whether aggregation of the periods of time during which the audio signal is produced is allowed based on a specimen collection protocol. When the timer reaches a value that is the same as, or greater than, the predetermined period of time based on the configuration of the device 100, 101, the PED will issue an alert, which can be one or more of audible, haptic, visual, and the like to alert the individual and/or the healthcare professional supervising specimen collection that specimen collection is complete. The container 140 can then be removed from the mixing chamber 120 and any necessary subsequent diagnostic techniques (e.g., lateral flow assay, polymerase chain reaction, etc.) can be performed to determine a presence of one or more exhalants of interest for which the capture material 150 is capable of capturing therein.

In some embodiments, the diagnostic test kit contains a reusable upper component (e.g., including the mouthpiece 160, mixing chamber 120, exhaust tube 180, and resonator 188) and multiple single-use containers 140. According to such an embodiment, the material and/or packaging waste can be reduced for instances in which a single diagnostic test kit can be used for testing a family unit and/or for repeated testing of one or more individuals to monitor, for example, disease progression.

In some embodiments, the device 100, 101 comprises removable and recoverable internal components, such as, for example and without limitation, a grid, spiral cone, porous design and material, etc. that can enhance binding of the gels (e.g., the capture material 150) for collection and recovery of the specimen, or immediate introduction in a detector device or for laboratory assay. Such internal components can be placed, for example, in the air flow path 10 to enable collection of the pathogen particles or of the analyte. Such internal components may be manufactured by any suitable manufacturing technique.

In some embodiments, the capture material 150 comprises a hydrogel for capturing an exhalant of interest (e.g., an infectious agent and/or particles thereof). The term “particles” is also meant to encompass intact infectious agents (such as a viral particle or prion particle) and particles or components or material derived from an infectious agent. In some embodiments of the device 100, 101, the hydrogel may be overlaid onto a conjugate pad following exposure to breath for the lateral flow portion of the assay. According to one example, it was successfully demonstrated that SARS-COV-2 was absorbed from a hydrogel onto a 4% agarose gel pad with a cellulose-based filter paper applied to the agarose pad, the filter pad extending beyond the agarose pad by about 1 centimeter (cm), for a period of 5 minutes. The agarose gel and filter paper can be, for example, incubated with medium to release virus. When this was performed, plaque assays were performed to measure viral titer. This technique has been used to demonstrate that the SARS-COV-2 virus was released from the agarose and drawn through the filter paper to the end of the filter paper, 1 cm beyond the agarose gel pad. In this example, the agarose gel pad had a viral load of 3.25e6 plaque forming units per milliliter (pfu/ml), the portion of the filter paper in contact with the agarose gel pad had a viral load of 2.25e6 pfu/ml, and the portion of the filter paper extending beyond (e.g., not in contact with) the agarose gel pad had a viral load of 7.5e5 pfu/ml. As such, the hydrogel is capable of releasing infectious agent particles for the lateral flow portion of the assay to enable the devices and systems disclosed herein to be used as a rapid point of care device that may not require processing in a laboratory to at least preliminarily diagnose an individual as being infected with a given infectious agent.

In some embodiments, the device may be combined with any desired assay, such as but not limited to downstream qRT-PCR assays, to detect a variety of infectious agents. RNA and/or DNA can be collected from the capture material 150, such as, for example, when the capture material 150 is or comprises a hydrogel, by directly adding an appropriate lysis buffer and following standard RNA and/or DNA isolation protocols. In one example, a series of tenfold dilutions of SARS-CoV-2 (5×104 to 50 PFU's) was applied to a hydrogel capture material 150 contained in the device 100 and was then allowed to absorb to the hydrogel. The RNA was isolated using an RNAqueous-Micro-Total RNA Isolation Kit (available from Invitrogen Corp.) according to the manufacturer's instructions. Since SARS-CoV-2 is an RNA virus, the lysis buffer used was an RNA lysis buffer, which was applied directly to the hydrogel of the capture material 150, and RNA was isolated, reverse transcribed to cDNA, and then analyzed for detection of SARS-CoV-2 using the GenArraytion COVID-19 Virus (SARS-CoV-2) Multiflex® PCR bioassay. This bioassay has a Ct threshold of 33; Ct values below 33 are considered positive. It was shown that as few as 50 PFUs applied to the capture material 150 can be detected using qRT-PCR in connection with the example embodiment of the device 100, 101 disclosed herein. For example, a 50×104 PFU sample had a mean Ct of 18.335, with a standard deviation of 0.057; a 50×103 PFU sample had a mean Ct of 22.478, with a standard deviation of 0.282; a 50×102 PFU sample had a mean Ct of 28/329, with a standard deviation of 0.062; and a 50 PFU sample had a mean Ct of 31.108, with a standard deviation of 0.638. In support of using exhaled breath for detection of SARS-COV-2, it is possible to collect exhaled breath condensate from individuals infected with SARS-COV-2. Since SARS-COV-2 mRNA is known to be present at the highest concentrations in specimens obtained during early stages of infection, it is particularly advantageous for the devices and systems disclosed herein to be early use as a rapid take home test using exhaled breath from potentially infected individuals to allow for diagnosis and quarantine of such individuals prior to becoming symptomatic to reduce the rate of transmission of SARS-COV-2 within the community.

A method of collecting a specimen is provided herein. The method comprises providing a device, system, and/or diagnostic test kit according to the disclosure herein, having an individual to be tested (e.g., a person who is suspected of being infected with the infectious agent, or analyte, of interest) blow for a predetermined amount of time, and/or for a specific number of individual breaths (e.g., 6, 12, or any desired quantity) into the mouthpiece so that the exhaled breath of the user comes into contact with a capture material contained within a removable container of the devices, systems, and/or diagnostic test kits. In one example, individuals were asked to breathe (e.g., forcibly exhale) into the device for 6 or 12 times and RNA was isolated directly from the hydrogel of the capture material. In this example, it was shown that RNA purity was insufficient for samples collected using only 6 breaths; however, when individuals exhaled into the device for 12 breaths, it was possible to obtain between 2-7 ng/μL of RNA that is of sufficient purity (e.g., A260/A280 ratio is close to 2.0) for use in downstream qRT-PCR assays, as described herein. In a first sample obtained using 12 breaths into the device, an RNA concentration of 7.61 ng/μL was obtained, with an A260/A280 ratio of 1.983. In a second sample obtained using 12 breaths into the device, an RNA concentration of 6.93 ng/μL was obtained, with an A260/A280 ratio of 1.837. In a third sample obtained using 12 breaths into the device, an RNA concentration of 3.769 ng/μL was obtained, with an A260/A280 ratio of 2.669. In a fourth sample obtained using 12 breaths into the device, an RNA concentration of 3.267 ng/μL was obtained, with an A260/A280 ratio of 2.155. In a fifth sample obtained using 12 breaths into the device, an RNA concentration of 2.264 ng/μL was obtained, with an A260/A280 ratio of 2.276. In a sixth sample obtained using 12 breaths into the device, an RNA concentration of 4.648 ng/μL was obtained, with an A260/A280 ratio of 1.998. The above example(s) is/are merely illustrative and the devices and methods disclosed herein can be used with greater or fewer number of breaths than the example breath quantities presented above, without limitation.

In some embodiments, the individual must blow into the mouthpiece to produce a sufficient volumetric flow rate of air from the outlet to produce an audible tone from the devices, systems, and/or diagnostic test kits for the predetermined amount of time. The container is then removed for further diagnostic testing techniques, including, for example, a lateral flow assay and/or polymerase chain reaction. The method then comprises reporting a result to the individual based on whether or not the exhalant of interest was present in the specimen collected.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A device for capturing exhaled breath of an individual, the device comprising:
a mouthpiece;
a mixing chamber, to which the mouthpiece is connected;
an exhaust tube connected to the mixing chamber, the exhaust tube comprising an outlet; and
a container having a capture material disposed therein;
wherein the mixing chamber is configured to cause exhaled breath from the individual introduced therein via the mouthpiece to contact the capture material and trap an exhalant in and/or on the capture material.

2. The device of claim 1, wherein the exhalant comprises an infectious agent and/or a particle thereof.

3. The device of claim 2, wherein the infectious agent is a virus, a bacterium and/or a fungus.

4. The device of claim 1, wherein the exhalant comprises a protein, polynucleotide, lipid, carbohydrate, and/or chemical biomarker indicating a presence of a disease, optionally the disease comprising a cancer or a metabolic disorder.

5. The device of claim 1, wherein the exhalant comprises an analyte indicating a presence of alcohol, tobacco, and/or other drugs.

6. The device of claim 1, wherein the container is mechanically and/or adhesively connected to the mixing chamber, optionally in a removable manner.

7. The device of claim 1, wherein the container is connected to the mixing chamber via a hinge.

8. The device of claim 1, wherein the capture material is directly linked to a detection assay.

9. The device of claim 8, wherein the detection assay comprises a colorimetric assay or a lateral flow assay.

10. The device of claim 1, comprising:
a flow restrictor positioned within the exhaust tube at a position adjacent to the outlet; and
a resonator connected to the exhaust tube at the outlet, the resonator comprising a resonator chamber that is shaped such that, as a flow of the exhaled breath through the exhaust tube is restricted by the flow restrictor and is emitted from the outlet, the device is configured to produce an audible tone when a predetermined volumetric flow rate of the exhaled breath is emitted from the outlet.

11. The device of claim 10, wherein the resonator chamber is closed by an end cap at an end of the resonator opposite the outlet.

12. The device of claim 10, wherein:
the mouthpiece is removably connected to the mixing chamber;
the exhaust tube is removably connected to the mixing chamber; and/or the resonator is removably connected to the exhaust tube.

13. The device of claim 12, wherein the mouthpiece, the mixing chamber, and the exhaust tube are formed integrally with each other, as a monolithic structure.

14. The device of claim 13, wherein the resonator is formed integrally with the exhaust tube, as a monolithic structure.

15. The device of claim 1, wherein the capture material comprises a gel or a hydrogel.

16. The device of claim 1, wherein the capture material is configured to release the exhalant captured therein for further diagnostic testing and/or wherein the capture material is configured for release from the container.

17. The device of claim 1, wherein the capture material comprises a gel of one or more of agarose, carbomers, Polyvinylalcohol (PVA), and polyacrylic acid (PAA).

18. The device of claim 1, wherein the capture material is a viscous fluid.

19. The device of claim 1, wherein the capture material is fixed onto an interior surface of the container in a manner of a coating on the interior surface of the container.

20. The device of claim 1, wherein the device is made via an additive manufacturing technique.

21. The device of claim 1, wherein the mouthpiece, mixing chamber, and exhaust tube are manufactured as a unitary structure via an injection molding technique.

22. The device of claim 1, wherein:
the capture material comprises an electric charge to increase capture of the exhalant by the capture material, relative to a non-electrically charged capture material;
the capture material is hydrophobic or hydrophilic to modify capture properties of the capture material;
the capture material is molded to increase a surface area of the capture material that is exposed to the exhalant within the device; and/or the capture material is in a form of a ball inside the device.

23. The device of claim 1, wherein the mixing chamber comprises an inner surface that is hydrophobic and/or negatively charged to inhibit the exhalant from adhering to the inner surface of the mixing chamber and to increase an amount of the exhalant that is captured by the capture material.

24. The device of any of claims 1-23, wherein the exhalant comprises SARS-COV-2 virus and/or particles thereof, an infectious agent or particles thereof associated with a Streptococcal infection, and/or an infectious agent or particles thereof associated with mononucleosis.

25. The device of any of claims 1-24, wherein the device is configured for diagnosis, monitoring, and/or study of conditions and/or diseases.

26. A system for detecting an exhalant contained within exhaled breath of an individual, the system comprising at least one device according to any of claims 1-25.

27. The system of claim 26, comprising a device, container, and reagent for recovering and/or analyzing the capture material and/or for recovering and/or analyzing the exhalant from the capture material and, optionally, a plurality of single-use containers, mouthpieces, and/or exhaust tubes.

28. A diagnostic test kit for detecting an exhalant contained within exhaled breath of an individual, the diagnostic test kit comprising at least one device according to any of claims 1-25.

29. The diagnostic test kit of claim 28, comprising a device, container, and reagent for recovering and/or analyzing the capture material and/or for recovering and/or analyzing the exhalant from the capture material and, optionally, a plurality of single-use containers, mouthpieces, and/or exhaust tubes.

30. A method for detecting an exhalant contained within exhaled breath of an individual, the method comprising:
providing at least one device according to any of claims 1-25;
flowing exhaled breath from the individual through the device for a predetermined period of time; and
capturing the exhalant in the capture material.

31. The method of claim 30, comprising producing an audible tone from the device when a sufficient volumetric flow rate of the exhaled breath is emitted through the outlet of the device.

32. The method of claim 31, comprising measuring a duration of the audible tone to determine that the sufficient volumetric flow rate of the exhaled breath is provided to the device for at least the predetermined period of time.

33. The method of claim 32, comprising removing the container from the mixing chamber and performing one or more further diagnostic testing techniques to detect whether the exhalant is contained within the capture material after the individual has provided the sufficient volumetric flow rate of the exhaled breath into the device for the predetermined period of time.

34. The method of claim 33, wherein the one or more further diagnostic testing techniques comprise a colorimetric assay or a lateral flow assay.

35. The method of claim 33, wherein the one or more further diagnostic testing techniques comprise polymerase chain reaction (PCR).
